(19) United States
(12) Patent Application Publication
(10) Pub. No.: US 2023/0263546 A1
(43) Pub. Date: Aug. 24, 2023

(54) MEDICAL MANIPULATOR AND TREATMENT METHOD

(71) Applicant: Olympus Corporation, Tokyo (JP)

(72) Inventor: Masahiro Fujii, Hachioji-shi (JP)

(21) Appl. No.: 18/306,734

(22) Filed: Apr. 25, 2023

Related U.S. Application Data
(63) Continuation of application No. PCT/JP20/40783, filed on Oct. 30, 2020.

(51) Int. Cl. A61B 17/29; A61B 18/12

(52) U.S. Cl. CPC A61B 17/29; A61B 18/12; A61B 2017/2926

(57) ABSTRACT

A medical manipulator includes: an elongated portion; an end effector provided to a distal end of the elongated portion and having a pair of jaws that open and close; and a manipulating portion provided to a proximal end of the elongated portion. One of the pair of jaws has a first member and a second member, the second member being rotatably attached to the first member. The other of the pair of jaws has a third member rotatably attached to the first member and the second member, a front portion of the third member being located to face the second member. The manipulating portion has a switching portion configured to switch between a first mode in which rotation of the second member with respect to the first member is restricted and a second mode in which rotation of the second member with respect to the first member is not restricted.

### CROSS REFERENCE TO RELATED APPLICATIONS

The present application is a continuation application based on PCT Patent Application No. PCT/JP2020/040783, filed on Oct. 30, 2020, the entire content of which is hereby incorporated by reference.

### BACKGROUND

### Technical Field

The present invention relates to a medical manipulator. A treatment method using the medical manipulator is also described.

### BACKGROUND

As a treatment for gastrointestinal tumors, there are procedures that do not perforate the duct wall, such as EMR (endoscopic mucosal resection) and ESD (endoscopic submucosal dissection), and full-thickness resection in which the area to be treated is excised along the thickness direction of the vessel wall. Full-thickness resection carries less risk of residual tumor than EMR or ESD.

Full-thickness resection has the advantages described above, but full-thickness resection creates a hole in the gastrointestinal tract, so it is necessary to close or seal the hole after tissue resection. Therefore, full-thickness resection requires various steps such as grasping, cutting, hemostasis, and suturing, and must be performed while exchanging treatment tools suitable for each step. Such procedure will be required when accessing a lesion from not only inside the gastrointestinal tract but outside the gastrointestinal tract.

In addition to full-thickness resection, there are procedures that require various types of processes, and these procedures also have similar problems.

In relation to this problem, Japanese Unexamined Patent Application, First Publication No. 2003-521304 (hereinafter referred to as Patent Document 1) discloses an instrument capable of performing two types of manipulations, grasping and cutting. In this instrument, a knife having a blade is housed in one of a pair of openable and closable jaws. One of the jaws containing the knife is elastically deformable, and when the jaw is deformed, the knife blade protrudes into contact with the tissue. Such a device has the potential to reduce the frequency of replacement of treatment tools in procedures that require many types of steps.

With the instrument described in Patent Document 1, the blade of the knife is pressed vertically against the tissue, so it is actually not easy to cut the tissue because the shear force cannot be applied to t the tissue efficiently. Since the magnitude of elastic deformation is difficult to control, there is a possibility that the blade of the knife may protrude unintentionally when grasping with a large amount of force, and the tissue may be cut at an unintended timing.

As described above, the instrument of Patent Document 1 does not sufficiently separate the grasping operation and the cutting operation.

### SUMMARY

In view of the above circumstances, an objective of the present invention is to provide a medical manipulator capable of performing a grasping operation and a cutting operation, and in which the grasping operation and the cutting operation are reliably separated.

A first aspect of the present invention is a medical manipulator including: an elongated portion; an end effector provided to a distal end of the elongated portion and having a pair of jaws that open and close; and a manipulating portion provided to a proximal end of the elongated portion.

One of the pair of jaws has a first member and a second member, the second member being rotatably attached to the first member.

The other of the pair of jaws has a third member rotatably attached to the first member and the second member, a front portion of the third member being located to face the second member.

The manipulating portion has a switching portion configured to switch between a first mode in which rotation of the second member with respect to the first member is restricted and a second mode in which rotation of the second member with respect to the first member is not restricted.

A second aspect of the present invention is a treatment method using a medical manipulator having an elongated portion, an end effector provided to a distal end of the elongated portion and having a pair of jaws that open and close, and a manipulating portion provided to a proximal end of the elongated portion, one of the pair of jaws having a first member and a second member, the second member being rotatably attached to the first member, the other of the pair of jaws having a third member rotatably attached to the first member and the second member, a front portion of the third member being located to face the second member, and the manipulating portion having a switching portion configured to switch between a first mode in which rotation of the second member with respect to the first member is restricted and a second mode in which rotation of the second member with respect to the first member is not restricted.

This treatment method includes: grasping tissue between the second member and the third member under the first mode; applying energy to the tissue grasped between the second member and the third member under the first mode to denature the tissue; and rotating the third member with respect to the first member under the second mode, to cut the tissue positioned between the third member and the first member by a shear force generated between the third member and the first member.

According to the present invention, both the grasping operation and the cutting operation can be performed with one manipulator while reliably separating the grasping operation and the cutting operation.

### BRIEF DESCRIPTION OF THE DRAWINGS

FIG. 1 is an overall view of a medical manipulator according to a first embodiment of the present invention.

FIG. 2 is an enlarged view of a distal end portion of the medical manipulator.

FIG. 3 is a front view of an end effector of the medical manipulator.

FIG. 4 is a diagram showing a process of using the medical manipulator.

FIG. 5 is a diagram showing a process of using the medical manipulator.

FIG. 6 is an enlarged view of the end effector in the process of FIG. 5.

FIG. 7 is a diagram showing a process of using the medical manipulator.

FIG. 8 is an enlarged view of the end effector in the process of FIG. 7.

FIG. 9 is an enlarged view of the distal end portion in a second mode.

FIG. 10 is a diagram showing a process of using the medical manipulator.

FIG. 11 is an enlarged view of the end effector in a process of FIG. 10.

FIG. 12 is a diagram showing a process of using the medical manipulator.

FIG. 13 is a flow chart showing an example of a procedure using the medical manipulator.

FIG. 14 is a schematic diagram of a rear portion of a stopper in a modified example of the medical manipulator.

FIG. 15 is an overall view of a medical manipulator according to a second embodiment of the invention.

FIG. 16A is a schematic diagram showing one mode at a time of mode switching in the medical manipulator.

FIG. 16B is a schematic diagram showing one form of mode switching in the medical manipulator.

FIG. 16C is a schematic diagram showing one form of mode switching in the medical manipulator.

FIG. 17 is a schematic diagram of a manipulating portion according to a modified example of the medical manipulator.

FIG. 18 is a diagram showing an operation when using the manipulating portion.

FIG. 19 is a schematic enlarged view of a distal end portion in a modified example of the present invention.

FIG. 20 is a front view of an end effector according to a modified example of the present invention.

FIG. 21 is a front view of an end effector according to a modified example of the present invention.

FIG. 22 is a front view of an end effector according to a modified example of the present invention.

### DETAILED DESCRIPTION

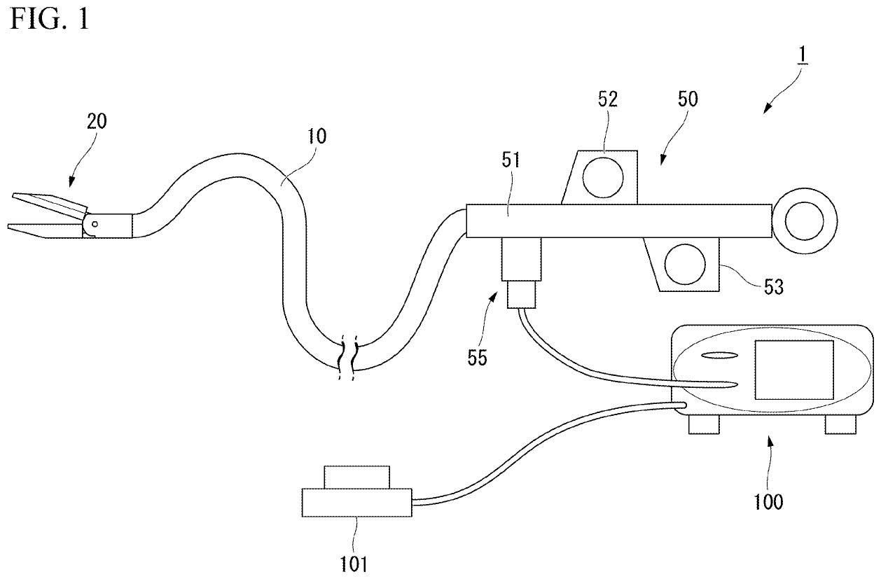

A first embodiment of the present invention will be described with reference to FIGS. 1 to 13.

FIG. 1 is an overall view of a medical manipulator (hereinafter simply referred to as “manipulator”) 1 of this embodiment. The manipulator 1 includes an elongated portion 10, an end effector 20 attached to a first end (distal end) of the elongated portion 10, an end effector 20 attached to a second end (proximal end) of the elongated portion 10, a manipulating portion 50, and an energy supply portion 100 connected to the manipulating portion 50.

The elongated portion 10 is made of a resin tube, a metal coil, or the like, and has flexibility. Wires for moving the end effector 20, wiring for supplying energy to the end effector 20, and the like are passed through the elongated portion 10.

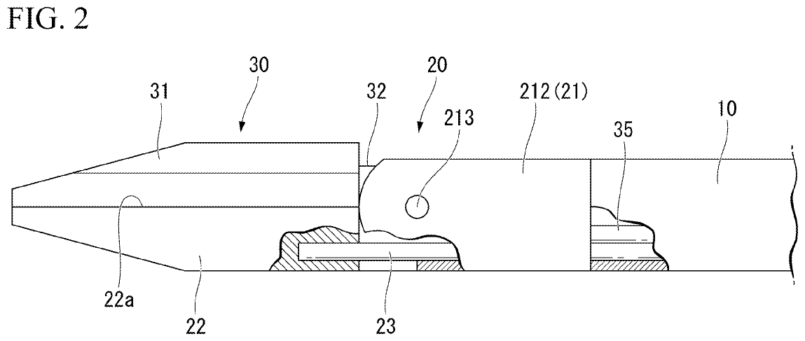

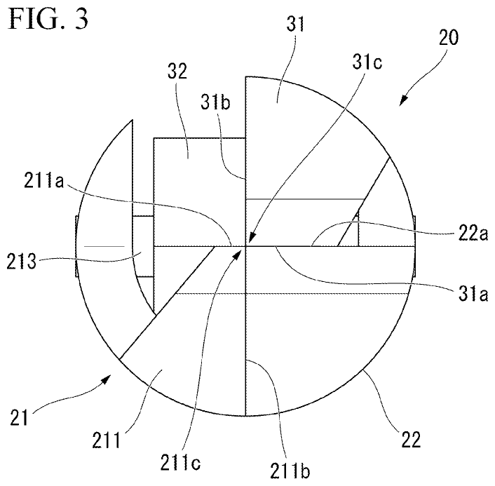

FIG. 2 is an enlarged view showing a partially exploded distal end portion of the manipulator 1 including the end effector 20. FIG. 3 is a front view of the end effector 20. The end effector 20 has a pair of jaws that can be opened and closed. The first jaw located on the lower side in FIG. 3 is composed of a first member 21 and a second member 22.

The first member 21 has a rear portion 212 connected to the elongated portion and a front portion 211 connected to the front side of the rear portion 212. In this embodiment, the front portion 211 constitutes the left side of the first jaw when viewed from the front. The rear portion 212 is cylindrical and connected to the rear side of the front portion 211. The rear portion 212 has two shaft holes through which a shaft member 213 is passed. The shaft member 213 is fixed to the rear portion 212 while extending into the internal space of the rear portion 212.

In this embodiment, the second member 22 constitutes the left side of the first jaw when viewed from the front. The second member 22 has a grasping surface 22a on the upper side, and the dimension in the vertical direction gradually decreases toward the front end. The second member 22 has a through-hole (not shown) extending in the left-right direction in the rear portion, and the shaft member 213 is passed through the through-hole. Thereby, the second member 22 is attached to the first member 21 so as to be relatively rotatable.

A bottomed hole extending forward is provided on the rear surface of the second member 22. A stopper 23 that limits the movement of the second member 22 enters this hole. The stopper 23 is, for example, a wire, and has sufficient rigidity to support the posture of the second member 22 without bending excessively during a grasping operation, which will be described later. The stopper 23 extends through the interior of the rear portion 212 and the elongated portion 10 to the manipulating portion 50.

The second jaw located on the upper side in FIG. 3 is composed of the third member 30. The third member 30 has a front portion 31 used for treatment and a rear portion 32 connected to the front portion 31.

The front portion 31 is located to face the front portion of the second member 22 and has a grasping surface 31a on the lower side. The rear portion 32 has a through-hole (not shown) extending in the left-right direction, and the shaft member 213 is passed through the through-hole. Thereby, the second member 22 is attached to the first member 21 and the second member 22 so as to be relatively rotatable. Since the rear portion 32 is offset from the front portion 31 in the front view of the end effector 20, the shaft member 213 can be passed through the through-hole without interfering with the second member 22 while arranging the front portion 31 to face the second member 22.

Although the width of the grasping surface 31a is smaller than the width of the grasping surface 22a in this embodiment, this is not essential in the present invention. The shape and dimensions of the grasping surface 31a and the grasping surface 22a can be appropriately set.

A power transmission member 35 for rotating the third member 30 is connected to the rear portion 32. The power transmission member 35 shown in FIG. 2 extends to the manipulating portion 50 through the interior of the rear portion 212 and the elongated portion 10. The power transmission member 35 may be attached directly to the rear portion 32 or may be attached to a pulley fixed to the rear portion 32.

The manipulating portion 50 has a main body 51 connected to the elongated portion 10 and a first slider 52 and a second slider (switching portion) 53 attached to the main body 51.

The main body 51 has a tubular shape, and the stopper 23 and the power transmission member 35 extending from the elongated portion 10 enter the main body 51.

The first slider 52 is slidably attached to the main body 51 and connected to the power transmission member 35 entering the main body 51. When the first slider 52 is slid with respect to the main body 51, the power transmission member 35 advances and retracts in the longitudinal direction, allowing the third member 30 to rotate around the shaft member 213.

The second slider 53 is slidably attached to the main body 51 and connected to the stopper 23 entering the main body 51. When the second slider 53 is slid with respect to the main body 51, the stopper 23 advances and retracts in the longitudinal direction, and the stopper 23 can be inserted into and removed from the hole provided on the rear surface of the second member 22.

In this embodiment, the energy supply portion 100 supplies high-frequency current. The energy supply portion 100 is connected to a plug 55 provided on the main body 51. Wiring (not shown) is connected to the plug. The wiring passes through the elongated portion 10 and is connected to the end effector 20. The energy supply portion 100 has a foot switch 101. By operating the foot switch 101, power supply to the end effector 20 can be turned on or off.

The connection mode between the energy supply portion 100 and the end effector 20 can be determined as appropriate. By connecting the energy supply portion 100 and the end effector 20 formed of a conductor with a single wire, the manipulator 1 can be configured as a monopolar high-frequency device. One of the two wires is connected to the third member 30, the other is connected to the first member 21 or the second member 22, and the first member 21, the second member 22, and the third member 30 are covered with insulation. The manipulator 1 can be configured as a bipolar type high-frequency device by arranging the manipulator 1 in a non-conducting state. If necessary, a portion of the outer surface of each member made of a conductor may be covered with an insulating coating to limit the portion to which energy can be applied.

A treatment method using the manipulator 1 configured as described above will be described using an example of full-thickness resection of the stomach wall.

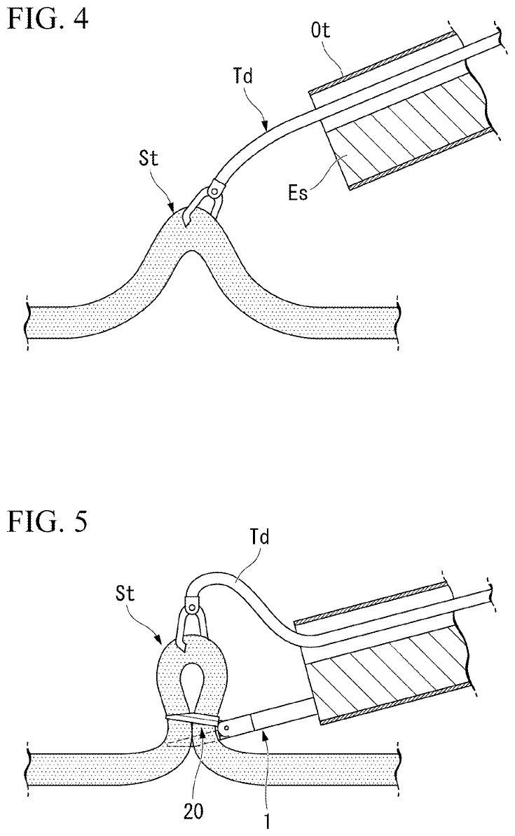

The operator introduces the endoscope and the traction instrument into the body, and pulls up the resection target tissue St toward the inside of the stomach with the traction instrument Td, as shown in FIG. 4. At this time, the tissue St is sufficiently pulled up so that the serosa just below the tissue St is higher than the surrounding mucous membrane (a position closer to the inside of the stomach).

Although FIG. 4 shows an example in which the endoscope Es and the traction device Td are passed through the overtube Ot and introduced into the body, the method of introducing the traction device is not limited to this way.

While maintaining the state in which the tissue St is pulled up, the operator pinches and grasps the base of the tissue St with the end effector 20 of the manipulator 1 as shown in FIG. 5 (step A). By this action, the stomach wall around the tissue St drawn by pulling up the tissue St comes into contact.

Step A is performed with the stopper 23 entering the hole of the second member 22. The operator operates the second slider 53 as necessary to insert the stopper 23 into the hole of the second member 22 to engage the second member 22 and the stopper 23. In the following description, this state is called “first mode”.

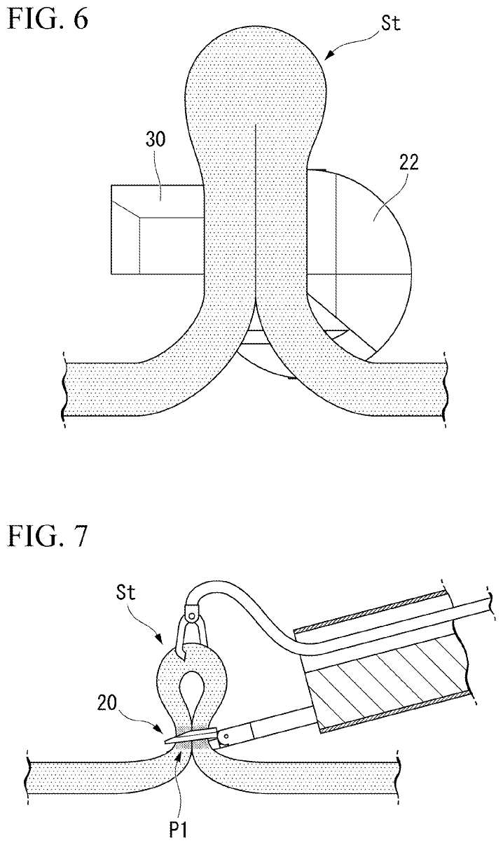

In the first mode, the third member 30 can rotate around the shaft member 213 by operating the first slider 52, but the second member 22 is supported by the stopper 23 and held in a state where rotation is restricted. Therefore, by rotating the third member 30 to approach the second member 22, the base of the tissue St located between the second member 22 and the third member 30 can be grasped as shown in FIG. 6, and the amount of grasping force can be increased.

The operator operates the foot switch 101 to apply energy to the part grasped by the end effector 20 (step B1). As a result, the grasped site is denatured and the stomach wall around the tissue St is joined.

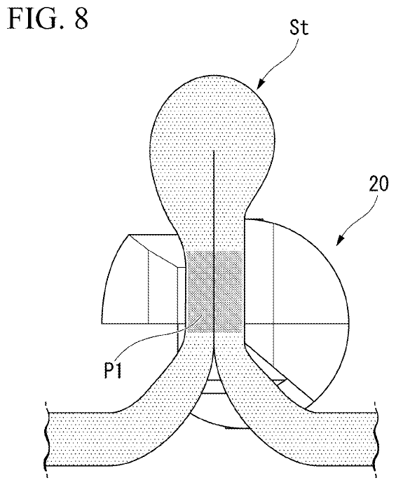

Subsequently, the operator operates the first slider 52 to increase the amount of grasping force of the end effector 20. As a result, as shown in FIGS. 7 and 8, the grasped portion P1 is brought into close contact with the pressure.

When the operator applies energy to the site P1 in this state (step B2), the blood in the blood vessel passing through the grasped site is coagulated. As a result, blood flow to the tissue St is blocked.

Steps B1 and B2 are also performed in the first mode.

The operator operates the second slider 53 to retract the stopper 23. When the stopper 23 is removed from the hole of the second member 22, the second member 22 is no longer supported by the stopper 23 and becomes rotatable with respect to the first member 21. In the following description, this state is called “second mode”.

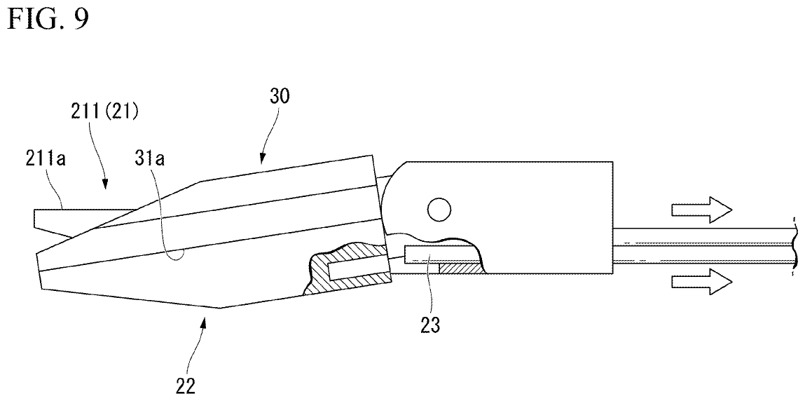

In the second mode, since the second member 22 is not supported by the stopper 23, the third member 30 can rotate further than in the first mode while pushing the second member 22. As a result, most of the grasping surface 31a can be moved below the upper surface 211a of the front portion 211 of the first member 21, as shown in FIG. 9.

As shown in FIG. 3, the left side surface 211b of the front portion 211 of the first member 21 is substantially perpendicular to the upper surface 211a and has a ridgeline 211c at the boundary between the upper surface 211a and the left side surface 211b. In the front portion 31 of the third member 30, the right side surface 31b is substantially perpendicular to the grasping surface 31a and has a ridgeline 31c at the boundary between the grasping surface 31a and the right side surface 31b. Furthermore, the distance between the left side 211b and the right side 31b is sufficiently small, and the left side 211b and the right side 31b are positioned on the same virtual plane.

When the third member 30 is rotated in the second mode, the ridgeline 31c and the ridgeline 211c intersect while coming into contact with each other from the rear side. Along with this, the right side surface 31b and the left side surface 211b move with respect to each other while being in close contact or sufficiently close to each other. As a result, a shear force is generated in the portion sandwiched between the first jaw and the second jaw, and the sandwiched portion is cut (that is, cold cut) without using the energy supplied from the energy supply portion 100 (Step C).

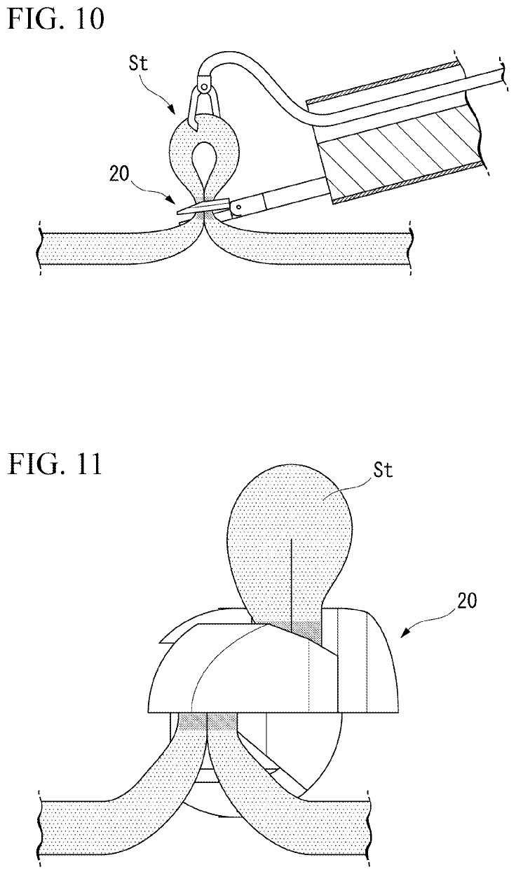

When step C is executed in a state where the root of the tissue St is sandwiched, a portion of the stomach including the tissue St located above the site sandwiched by the end effector 20 is resected over the entire thickness, as shown in FIGS. 10 and 11.

At this time, since the blood flow to the tissue St is blocked by step B2, bleeding does not occur. Since the stomach wall around the tissue St is joined by step B2, no hole is formed in the stomach.

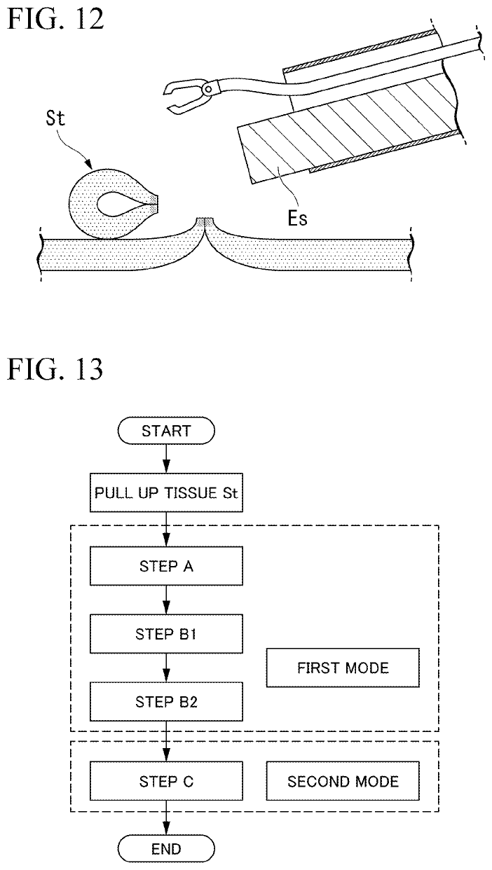

As shown in FIG. 12, the operator uses the endoscope Es to check the joint state of the gastric wall at the site where the procedure was performed, and ends the procedure if there is no problem. If necessary, sutures, clips, staples, etc. may be used to reinforce the junction of the stomach walls.

In the conventional procedure, the step A of grasping the tissue, the steps B1 and B2 of applying energy to the tissue, and the step C of cold-cutting the tissue were performed using different treatment instruments. Therefore, in order to perform the series of procedures described above, it was necessary to frequently replace the treatment instrument.

With the manipulator 1 of the present embodiment, all these steps can be performed as described above, so the replacement frequency of treatment tools is significantly reduced. As a result, it can greatly contribute to shortening the operation time and reducing the burden on the patient.

The first mode in which the rotation of the second member 22 is restricted and the second mode in which the rotation of the second member 22 is not restricted can be easily switched by operating the manipulating portion 50.

In the first mode, even if a large rotational force is applied to the third member in an attempt to increase the amount of grasping force, the second member 22 whose rotation is restricted by the stopper 23 receives this force. Therefore, the grasping surface 31a does not move below the ridgeline 211c of the first member 21. Therefore, in the first mode, it is possible to reliably suppress the occurrence of unintended shear force on the tissue sandwiched between the end effectors 20, and it is easy to adjust the amount of grasping force to a size suitable for the tissue.

On the other hand, in the second mode, tissue can be cut without using energy due to the shear force described above, so unnecessary damage to surrounding tissue can be suppressed when cutting tissue.

That is, in this embodiment, the operation of grasping the tissue and the operation of cold-cutting the tissue are completely separated, and the possibility of unintentionally cold-cutting the tissue while grasping the tissue is minimized to nearly zero.

FIG. 13 is a flowchart showing the flow of the series of procedures described above. This flowchart includes the treatment method according to this embodiment. As shown in FIG. 13, this procedure includes steps performed in the first mode and steps performed in the second mode. By using the manipulator 1, switching between the first mode and the second mode can be done smoothly.

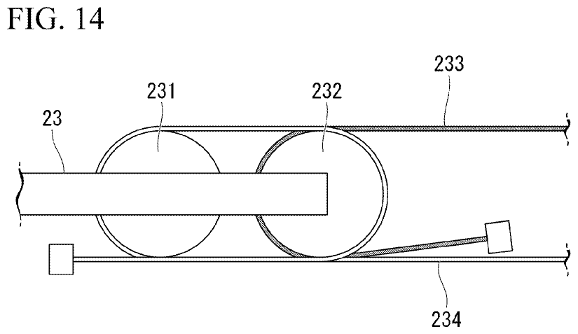

In the present embodiment, an example in which the stopper 23 extends to the manipulating portion has been described, but the stopper 23 may end in the middle of the elongated portion 10. In the schematic diagram of the modified example shown in FIG. 14, two pulleys 231 and 232 are attached to the rear end of the stopper 23. Two drive wires are wound around the pulley and extend to the manipulating portion 50.

When the drive wire 233 wound only on the pulley 232 on the rear side is pulled, the stopper 23 moves backward, and when the drive wire 234 wound on both pulleys 231 and 232 is pulled, the stopper 23 moves forward.

Even with such a configuration, it is possible to switch between the first mode and the second mode by moving the stopper 23.

In addition, even if the rear end of the stopper is threaded to change the fitting length or a rack-and-pinion mechanism is provided at the rear end of the stopper, it is possible to switch between the first mode and the second mode by advancing and retracting the stopper.

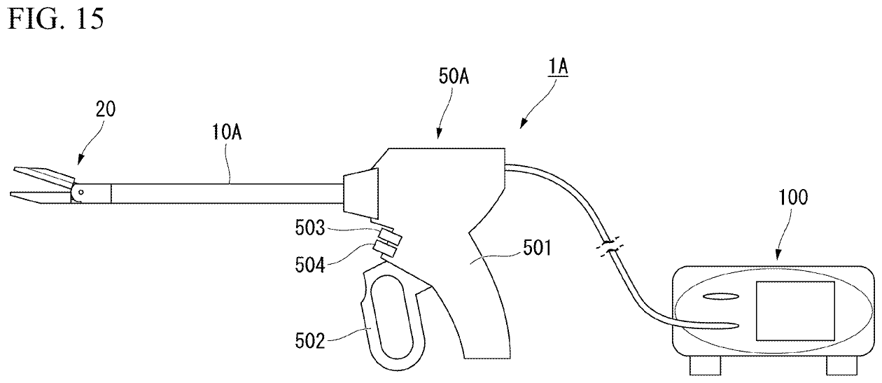

A second embodiment of the present invention will be described with reference to FIGS. 15 to 18. In the following description, the same reference numerals are given to the same configurations as those already described, and redundant descriptions will be omitted.

A manipulator 1A of the present embodiment shown in FIG. 15 is a rigid treatment instrument used for laparoscopic surgery or the like. The manipulator 1A includes an elongated portion 10A instead of the elongated portion 10, and a manipulating portion 50A instead of the manipulating portion 50.

The elongated portion 10A is a rigid pipe that does not have flexibility. The stopper and the power transmission member passed through the elongated portion 10A are rods that are thicker and harder to bend than those of the first embodiment, and are easy to advance and retract.

The manipulating portion 50A includes a main body 501, a handle 502 attached to the main body 501, a first switch 503, and a second switch (switching portion) 504.

A power transmission member is connected to the handle 502. By operating the handle 502, the third member 30 can be rotated by moving the power transmission member forward and backward.

The first switch 503 has the function of switching on/off the energy supply, like the foot switch 101 of the first embodiment.

The second switch 504 is used when switching between the first mode and the second mode.

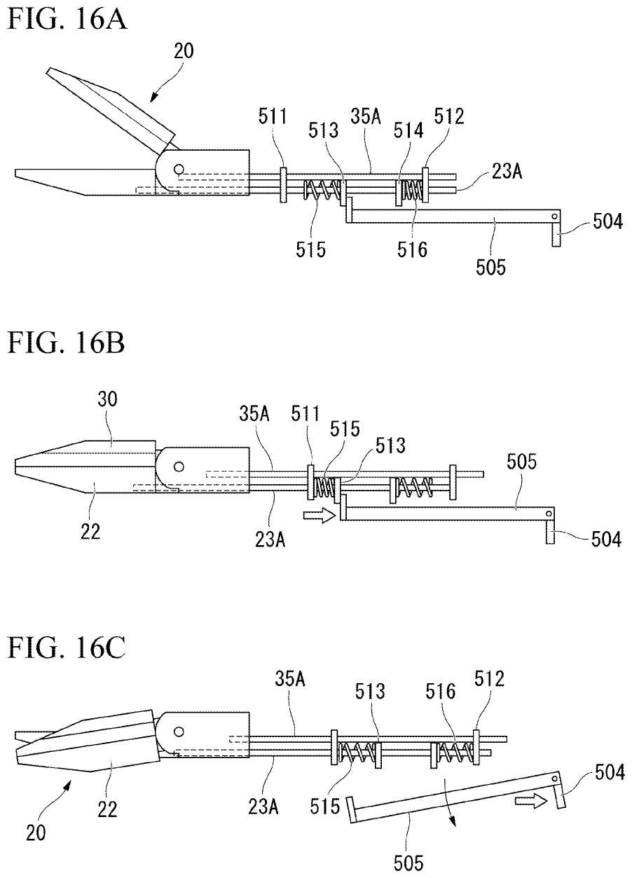

The operation of each part at the time of mode switching of the manipulator 1A will be described with reference to the schematic diagrams of FIGS. 16A to 16C.

As shown in FIG. 16A, two plates, a first plate 511 on the distal side and a second plate 512 on the proximal side, are fixed to the power transmission member 35A. Two plates, a third plate 513 on the distal side and a fourth plate 514 on the proximal side, are also fixed to the stopper 23A.

The third plate 513 is positioned closer to the proximal side than the first plate 511, and the first spring 515 is attached to the distal side. The fourth plate 514 is positioned distally with respect to the second plate 512 and has a second spring 516 attached proximally.

A supporter 505 that restricts the movement of the stopper 23A is connected to the second switch 504. When the second switch 504 is not pressed, the distal end of the supporter 505 is positioned proximal to the third plate 513 by a torsion spring or the like, and contacts the third plate 513 from the proximal side.

When the operator grasps the handle 502 with the end effector 20 open as shown in FIG. 16A, the power transmission member 35A retracts and the third member 30 rotates in a direction approaching the second member 22. As a result, the first plate 511 contacts the first spring 515. The first plate 511 compresses the first spring 515, but since the third plate 513 is supported by the supporter 505, the stopper 23A does not retract. Therefore, when the second switch 504 is not pressed, the first spring 515 is compressed while the first mode is maintained.

When the operator presses the second switch 504 in the state shown in FIG. 16B, the supporter 505 rotates as shown in FIG. 16C. As a result, the support of the third plate 513 by the supporter 505 is released, and the compression of the first spring 515 is released. As a result, the stopper 23A retracts and exits the hole of the second member 22, switching the mode of the manipulator 1A to the second mode.

When switching the second mode to the first mode, the handle 502 is operated to advance the power transmission member 35A. When the power transmission member 35A advances, the second plate 512 contacts the second spring 516. After that, the power transmission member 35A and the stopper 23A move forward together, and the stopper 23A enters the hole of the second member 22 while the end effector 20 opens.

Also in the manipulator 1A of this embodiment, as in the first embodiment, the operation of grasping the tissue and the operation of cold-cutting the tissue can be performed while being completely separated. Therefore, for example, by grasping a blood vessel, applying energy to coagulate it, and then performing a cold cut, it is possible to easily excise the blood vessel or tissue without causing bleeding.

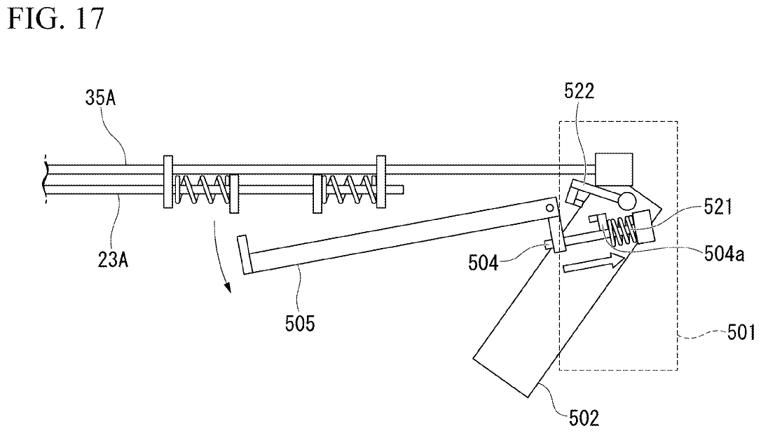

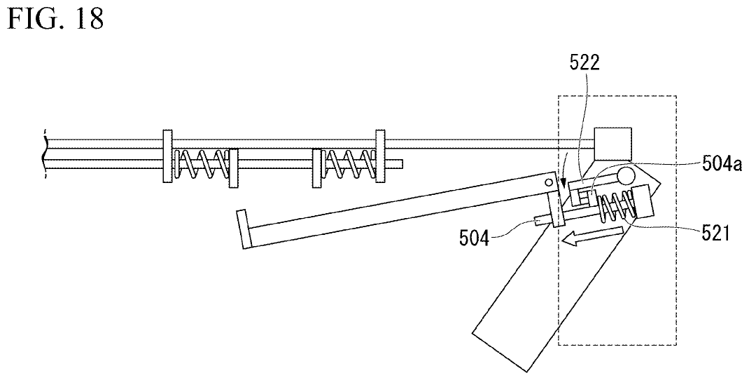

In this embodiment, a mechanism may be provided to keep the second switch 504 pressed. FIGS. 17 and 18 show an example of an operating section having such a mechanism.

A spring 521 is attached to the second switch 504 as shown in FIG. 17. One end of the spring 521 is fixed to the handle 502 inside the main body 501. Additionally, a lock 522 is pivotally attached to the handle 502. The second switch 504 is provided with a protrusion 504a that engages with the lock 522.

When the second switch 504 is pushed and the supporter 505 is rotated, the spring 521 is compressed by the pushed second switch 504. When the user rotates the lock 522 in this state, as shown in FIG. 18, the distal end of the lock 522 moves forward of the protrusion 504a. When the user releases the second switch 504, the second switch 504 moves slightly forward due to the restoring force of the spring 521. As a result, the protrusion 504a engages the lock 522.

Since the second switch 504 is biased forward by the spring 521, the second switch 504 does not naturally retract and disengage from the lock 522, and the state in which the second switch 504 is pushed is preferably maintained. The engagement between the second switch 504 and the lock 522 can be released by returning the lock 522 to its original position while pushing the second switch 504.

The manipulator 1A according to the present embodiment is provided with such a manipulating portion, so that tissue cutting in the second mode can be continuously performed without continuously pressing the second switch.

Although each embodiment of the present invention has been described above, the technical scope of the present invention is not limited to the above-described embodiments. It is possible to change the combination of components, add various changes to each component, or delete them without departing from the scope of the present invention. Below are some examples of changes, but they are not exhaustive. Also, two or more of these can be combined as appropriate.

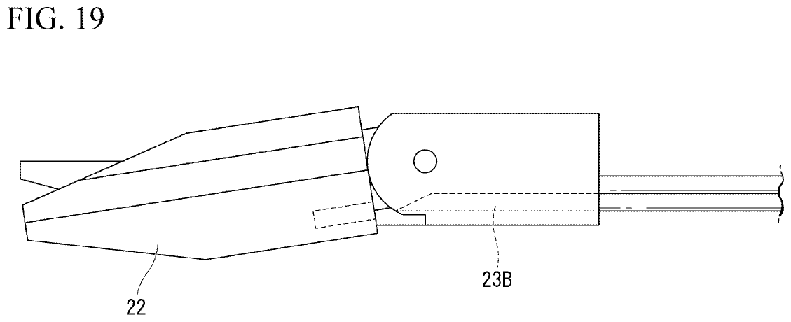

As with the stopper 23B shown in FIG. 19, the stopper may be tapered toward the distal end. In the second mode, the rotation of the second member 22 may form an angle between the stopper and the hole into which the stopper enters. However, if the stopper is tapered, the stopper can easily enter the hole even in such a state and smoothly return to the first mode.

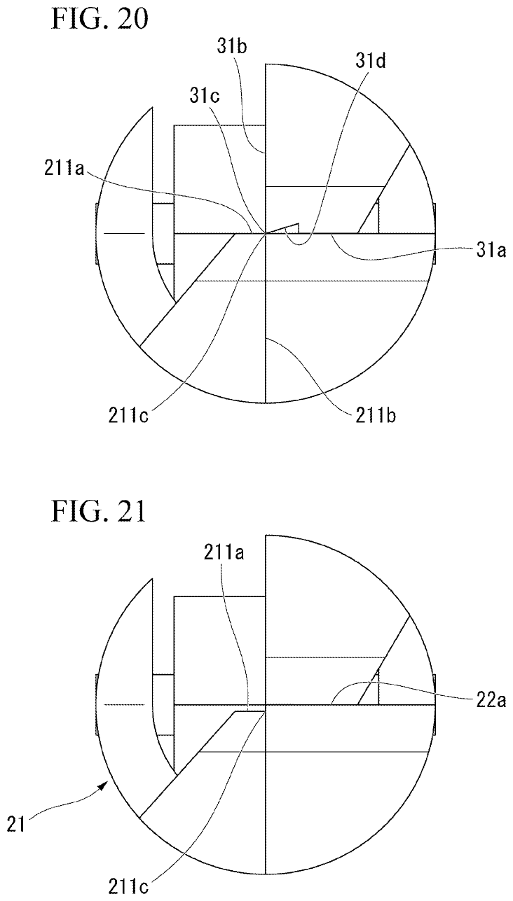

The third member may have a blade surface 31d forming an acute angle with the right side surface 31b between the ridge line 31c and the grasping surface 31a, as in the modification shown in FIG. 20. With this configuration, in the second mode, the generated shear force is increased, and the cold cut performance is improved.

Similarly, in the front portion of the first member, the upper surface 211a connected to the ridge line 211c may be formed to form an acute angle with the left side surface 211b, so that the upper surface may be used as a cutting surface.

The position of the grasping surface 22a in the first mode may be higher than the upper surface 211a of the first member 21, as in the modification shown in FIG. 21. With this configuration, grasping in the first mode is performed at a position higher than the ridgeline 211c, so the possibility of shear force acting on the tissue can be further reduced.

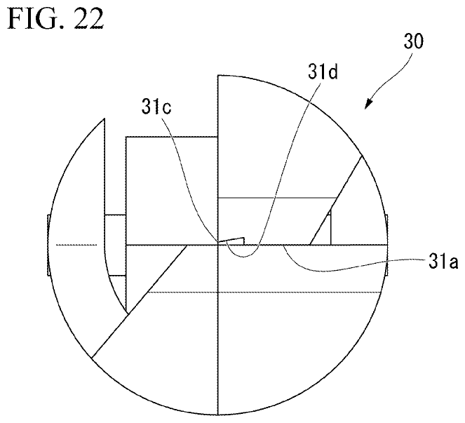

As in the modification shown in FIG. 22, in the third member 30, the position of the blade surface 31d may be higher than the grasping surface 31a. With this configuration, grasping in the first mode is performed at a position lower than the ridgeline 31c, so that the possibility of shear force acting on the tissue can be further reduced.

In the end effector, the second member and the third member may be located on the left side when viewed from the front. Such a configuration can be realized by horizontally reversing the structure shown in FIG. 3 and the like.

The present invention can be applied to a medical manipulator.

What is claimed is:

1. A medical manipulator, comprising:
an elongated portion;
an end effector provided to a distal end of the elongated portion and having a pair of jaws configured to open and close; and
a manipulating portion provided to a proximal end of the elongated portion;
wherein one of the pair of jaws has a first member and a second member, the second member being rotatably attached to the first member,
the other of the pair of jaws has a third member rotatably attached to the first member and the second member, a front portion of the third member being located to face the second member, and
the manipulating portion has a switching portion configured to switch between a first mode in which rotation of the second member with respect to the first member is restricted and a second mode in which rotation of the second member with respect to the first member is not restricted.

2. The medical manipulator according to claim 1, further comprising:
a stopper configured to engage the second member,
wherein the switching portion is configured to move the stopper, and
in the first mode, the second member is held to the first member so as not to rotate by engaging the stopper.

3. The medical manipulator according to claim 1, wherein
each of the first member and the third member has a ridgeline located on a same virtual plane, and
by rotating the third member with respect to the first member in the second mode, the first member and the third member are configured to cut a tissue sandwiched between the first member and the third member by a shear force.

4. The medical manipulator according to claim 1, wherein
each of the second member and the third member each a grasping surface located to face each other, and
by rotating the third member with respect to the second member in the first mode, the first member and the third member are configured to grasp a tissue.

5. The medical manipulator according to claim 4, wherein
the end effector includes a conductor and is configured to apply energy, and
the medical manipulator is configured such that the tissue grasped in the first mode can be denatured by the energy.

6. The medical manipulator according to claim 3, wherein
one of the first member and the third member has a blade surface that continues to the ridgeline.

7. A treatment method using a medical manipulator having a pair of jaws provided to a distal end of an elongated portion, one of the pair of jaws having a first member, a second member and a third member, the treatment method comprising:
grasping tissue between the second member and the third member under a first mode, the second member being rotatably attached to the first member, and the third member being rotatably attached to the first member and the second member, wherein the first mode is that the second member is configured to rotate with respect to the first member is restricted;
applying energy to the tissue grasped between the second member and the third member under the first mode to denature the tissue; and the
rotating the third member with respect to the first member under a second mode, to cut the tissue positioned between the third member and the first member by a shear force generated between the third member and the first member, wherein the second mode is that the second member is configured to rotate with respect to the first member is not restricted.
